(19) United States
(12) Patent Application Publication
(10) Pub. No.: US 2023/0028798 A1
(43) Pub. Date: Jan. 26, 2023

(54) ADIPOSE TISSUE CRYOPRESERVATION PROTECTIVE AGENT

(71) Applicant: Shanghai Ninth People's Hospital, Shanghai JiaoTong University School of Medicine, Shanghai (CN)

(72) Inventors: Qingfeng LI, Shanghai (CN); Shuangbai ZHOU, Shanghai (CN)

(73) Assignee: Shanghai Ninth People's Hospital, Shanghai JiaoTong University School of Medicine, Shanghai (CN)

(21) Appl. No.: 17/787,948

(22) PCT Filed: Dec. 21, 2020
(86) PCT No.: PCT/CN2020/137926

§ 371 Date: Jun. 22, 2022

(30) Foreign Application Priority Data
Dec. 26, 2019 (CN) 201911363434.7

(51) Int. Cl. A01N 1/02; C12N 5/077

(52) U.S. Cl. CPC A01N 1/0221; C12N 5/0653

(57) ABSTRACT

Use of glycerol in preparing an adipose tissue cryopreservation protective agent and the adipose tissue cryopreservation protective agent. Based on the total volume of the adipose tissue cryopreservation protective agent, the adipose tissue cryopreservation protective agent includes: glycerol with a volume fraction of 60-80%; 0.090-0.200 g/ml trehalose; and a PBS buffer serving as the solvent. The adipose tissue cryopreservation protective agent is free of DMSO and other protective agents having biological toxicity, poses no toxicity to cells and tissues, and causes no safety problem during clinical use even in a case of incomplete elution. The adipose tissue cryopreservation protective agent is free of xenoantigen and has low cost. With glycerol and trehalose being clinically approved medicinal ingredients and inexpensive products, the agent of the present disclosure contains no costly products such as human albumin, thus providing increased possibility for clinical use and promotion.

### TECHNICAL FIELD

The present disclosure relates to the field of medicine, and in particular, to an adipose tissue cryopreservation protective agent.

### BACKGROUND

With the continuous development of fat transplantation technology, autologous fat transplantation technology has been widely used in clinical practice, such as scar filling, facial atrophy, tissue reconstruction, wrinkles and breast filling. However, fat transplantation has the problem of high reabsorption. In most cases, repeated liposuctions, as well as fat filling immediately after the liposuctions, are still required to achieve accurate repair. Since liposuctions involve severe local damage, which causes great pain to the patient, local compression is required for at least 1 month after each liposuction procedure. In addition, liposuction has a high risk of infection. Therefore, if the extracted fat can be effectively stored for a long time and fat filling treatment is performed when necessary, the times of surgery for fat extraction can be reduced, which will significantly reduce the intraoperative pain of patients, reduce the time for postoperative recovery, lower the financial burden of surgery and surgical risks, and improve the efficiency of treatment.

Cryopreservation is a method that is capable of preserving cells and tissues for a long time. During the freezing process, cryopreservation protective agents are usually added to protect cells from damage caused by the crystallization of water molecules at low temperatures.

However, the effectiveness of adipose tissue cryoprotection is still controversial, with problems such as reduced tissue activity after the freezing process and low tissue retention rate after retransplantation. Further, traditional tissue cryopreservation protective agents contain biotoxic substances or xenoantigens, which pose biosafety problems after retransplantation. No effective, safe and non-toxic cryopreservation protective agent has been found for long-term cryopreservation of adipose tissue. Compared with simple cell suspension or other homogeneous tissues, adipose tissue has higher requirements for cryopreservation protective agents.

### SUMMARY

The present disclosure provides an adipose tissue cryopreservation protective agent.

A first aspect of the present disclosure provides the use of glycerol for the preparation of adipose tissue cryopreservation protective agent.

A second aspect of the present disclosure provides an adipose tissue cryopreservation protective agent, which includes, based on the total volume of the adipose tissue cryopreservation protective agent, the following components:

glycerol with a volume fraction of 60-80%;

0.090-0.200 g/ml of trehalose; and

a PBS buffer serving as the solvent.

A third aspect of the present disclosure provides a method for preparing an adipose tissue cryopreservation protective agent, including the following operations:

1) adding trehalose into PBS buffer to prepare trehalose solution;

2) preparing the adipose tissue cryopreservation protective agent using glycerol as the solute and the trehalose solution as the solvent.

A fourth aspect of the present disclosure provides a method for cryopreservation of adipose tissue, including the following operations:

1) mixing the adipose tissue cryopreservation protective agent with an adipose tissue at a volume ratio of 1:1 to obtain a mixture 1;

2) placing the mixture 1 in a programmed cooling box and performing programmed cooling at −80° C.; and

3) placing the resulting mixture 1 obtained in operation 2) in liquid nitrogen for cryopreservation, to obtain a cryopreserved adipose tissue.

A fifth aspect of the present disclosure provides a method for rewarming cryopreserved adipose tissue, including the following operations:

a) taking out the cryopreserved adipose tissue from liquid nitrogen, and heating the adipose tissue in water bath;

b) eluting the adipose tissue obtained in operation a) with PBS buffer, and centrifuging for a first time; and

c) collecting the upper-layer substance obtained after the centrifuging for the first time, eluting with PBS buffer, and centrifuging for a second time, and the upper-layer substance obtained after the centrifuging for the second time is the rewarmed adipose tissue.

As described above, the adipose tissue cryopreservation protective agent of the present disclosure has the following beneficial effects:

The adipose tissue cryopreservation protective agent is free of DMSO and other protective agents having biological toxicity, poses no toxicity to cells and tissues, and causes no safety problem during clinical use even in a case of incomplete elution. The adipose tissue cryopreservation protective agent of the present disclosure is free of xenoantigen, which may cause hypersensitivity reactions after fat transplantation when fetal bovine serum is used in cryopreservation protective agent, whereas there are no such biological agents in the present disclosure. The adipose tissue cryopreservation protective agent of the present disclosure also has low cost with glycerol and trehalose being clinically approved medicinal ingredients and inexpensive products, the agent of the present disclosure contains no costly products such as human albumin, thus providing increased possibility for clinical use and promotion. Strong protection is provided, the activity of adipose tissues is high when they are revived, and less inflammatory cells are formed after a transplantation.

### BRIEF DESCRIPTION OF THE DRAWINGS

FIG. 1 shows a comparison of adipose cells and adipose tissue. The left image shows the microscopic picture of the cells, and the right image shows the adipose tissue (HE staining). It can be seen that compared with cells, adipose tissue has a large mass, a large number of cells, various types of cells, and has an inherent organizational structure. The cell suspension, on the other hand, contains only a single species of cells, which are more dispersed without obvious clumps, and the cryopreservation protective agent is more likely to encapsulate each cell.

FIG. 2 shows a diagram of a fat transplantation performed on the back of a nude mouse. A: Fat transplantation was performed after the adipose tissues were cryopreserved under the protection of the present disclosure: the tissue block was seen to be surrounded by peripheral vessels, and the transplanted fat was homogeneous, with no obvious areas of fat liquefaction or necrosis.

B: Fat transplantation was performed after the adipose tissues were cryopreserved under the protection of FBS+DMSO: the tissue block was still vascularly surrounded, but areas of liquefaction or necrosis were visible at both upper and lower parts.

The comparison between A and B shows that by using the cryopreservation protective agent of the present disclosure, the tissue blocks transplanted after cryopreservation of adipose tissue are generally in better morphology.

FIG. 3 shows the results of G3PDH assay of various samples; G3PDH is an assay for quantitative measurement of tissue block activity.

FIG. 4 shows the retention rate of fat transplantation after cryopreservation at −196° C.

FIG. 5 shows the HE staining results of sliced adipose tissues, where these adipose tissues were transplanted to the back of nude mice after being cryopreserved at −196° C. for 6 months, and then these transplanted adipose tissues were observed for one month. (A: result diagram of fat after being cryopreserved without adding any cryopreservation protective agent; B: result diagram of fat after being cryopreserved under the protection of FBS+DMSO; C: result diagram of fat after being cryopreserved under the protection of 0.25 mol/L trehalose; D: result diagram of fat after being cryopreserved under the protection of the cryopreservation protective agent described in Embodiment 1; in the figure, * represents fibrosis tissue, A represents inflammatory infiltration, # represents vacuole after fat necrosis).

### DETAILED DESCRIPTION

Traditional cell cryopreservation protective agents mostly aim at a single type of cell and are less effective in preserving composite tissues such as adipose tissue. Cryopreservation protective agents that are effective for cryoprotection of single-type cells are less effective in preserving the three-dimensional structure of tissues when preserving composite tissues. Cell suspensions treated with cryopreservation protective agents and then cryogenically frozen usually have an activity of 95% or above after thawing, while tissues treated with conventional cryopreservation protective agents usually only have an activity of about 30%.

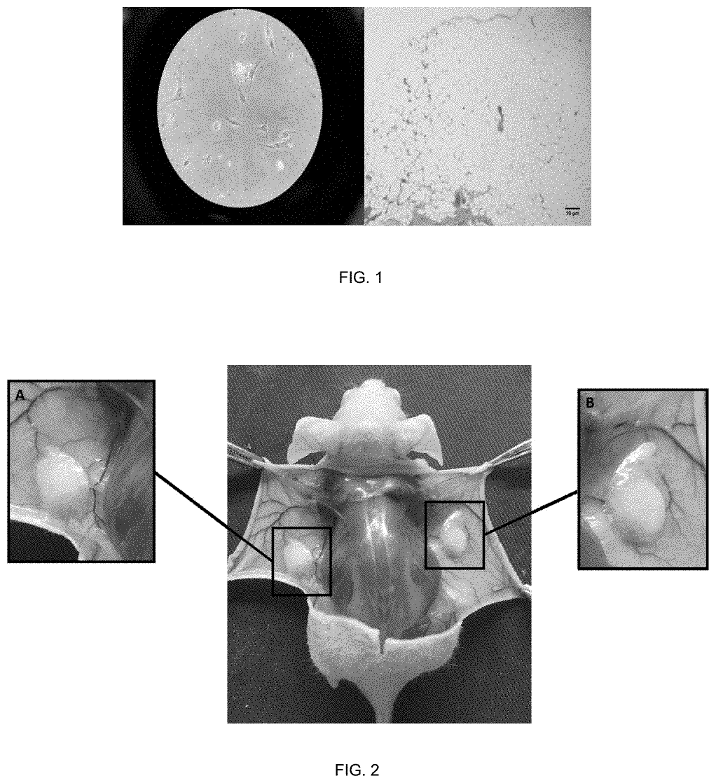

As a composite tissue, adipose tissue contains many kinds of cells, including adipocytes, adipose precursor cells, adipose stem cells, fibroblasts, and vascular endothelial cells (FIG. 1). Moreover, adipose tissue clumps are large in size, making it difficult for traditional cryopreservation protective agents to effectively penetrate the tissue. Adipose tissue is mainly composed of adipocytes, which have fewer organelle components and are mostly triglyceride-rich lipid droplets. Therefore, adipose tissue differs significantly from other cells and tissues. As a result, cryopreservation protective agents commonly used for other tissues can hardly provide effective protection for the specific structure and components of the adipose tissue. Secondly, protective agents penetrating into the interior of the adipose tissue are often difficult to be fully removed by elution. Cryopreservation protective agents formulated with fetal bovine serum (FBS) and dimethyl sulfoxide (DMSO) contain xenoantigens and cytotoxic DMSO. Therefore, residuals of these protective agents may cause toxicity and allergic reactions in patients during clinical use (according to Sigma product number Vetec-V900090 instructions). Effective cryopreservation of adipose tissue requires not only good morphology at the moment of taking out, but also the biological activity of the cells within the tissue, to ensure its survival rate and biological function after retransplanting and reduce tissue destruction caused by local inflammatory reactions. Therefore, so far there is still no effective and non-toxic cryopreservation protective agent for long-term cryopreservation of adipose tissue that is cost-effective and suitable for mass promotion.

Glycerin is an osmotic cryopreservation protective agent that can exert cryoprotective effects at low temperatures through mechanisms such as inhibition of ice crystal formation, prevention of osmotic damage, and protection of cell membranes and intracellular proteins. Glycerol is currently being studied in the cryopreservation of composite tissues such as skin, cartilage, and testes. In terms of adipose tissue preservation, glycerol is similar to triglycerides abundant in adipose cells, which can better preserve the activity of adipose cells and the integrity of lipid droplets. Moreover, it has been verified at the cytological level that infiltrating adipose cells in glycerol solution can effectively inhibit the efflux of triglyceride from adipose cells. Glycerol is highly osmotic to composite tissues and can better penetrate and be eluted from adipose tissues with larger clumps. Further, glycerol is non-toxic to tissues and has a low cost, which is conducive to mass clinical use. Therefore, the cryopreservation protective agent with glycerol as the main active ingredient can effectively protect the tissue activity of fat under cold storage conditions and reduce apoptosis. The resuscitation process is free from the toxic side effects of protective agent residues, and the morphological structure of the transplanted tissue is maintained well. In the present disclosure, a certain concentration of trehalose is further added as a non-osmotic cryopreservation protective agent to enhance the protective effect on tissues.

Cell cryopreservation protective agents using trehalose alone as the main ingredient are now available, but they mostly aim to protect the cell suspension rather than adipose tissues. In the present disclosure, trehalose alone served as a control for verification, and it was found that the morphology of the transplanted tissue of the group added with glycerol was significantly better than that of the group containing only trehalose. The transplanted tissue protected only by trehalose showed obvious infiltration of inflammatory cells, which may result from the immune response caused by the necrosis of adipose cells due to the poor protection effect under cryopreservation. In contrast, experiments under the same conditions showed that after being treated with the cryopreservation protective agents of the present disclosure, the transplanted adipose tissue has significantly less inflammatory infiltration, providing solid evidence that adipose tissue cryopreservation protective agents with glycerol as the main component are superior.

The embodiments of the present disclosure will be described below. Those skilled in the art can easily understand other advantages and effects of the present disclosure according to the contents disclosed by the specification. The present disclosure may also be implemented or applied through other different specific implementation modes. Various modifications or changes may be made to all details in the specification based on different points of view and applications without departing from the spirit of the present disclosure.

The present disclosure provides the use of glycerol in preparing an adipose tissue cryopreservation protective agent.

In the adipose tissue cryopreservation protective agent, glycerol serves as an osmotic protective agent.

Further, glycerol serves as an effective ingredient of the adipose tissue cryopreservation protective agent.

Further, the adipose tissue cryopreservation protective agent is free of dimethyl sulfoxide, animal serum, or human albumin.

In one embodiment, the adipose tissue cryopreservation protective agent further includes trehalose and PBS buffer.

In the adipose tissue cryopreservation protective agent, trehalose serves as an auxiliary non-osmotic protective agent, to more comprehensively reduce the formation of ice crystals and their damage to cell structure during the cryopreservation process.

In one embodiment, the adipose tissue cryopreservation protective agent includes, based on the total volume of the adipose tissue cryopreservation protective agent, the following components:

glycerol with a volume fraction of 60-80%;

0.090-0.200 g/ml of trehalose; and

a PBS buffer serving as the solvent.

In one embodiment, the content of the trehalose is 0.25-0.50 mol/L. Optionally, the content of the trehalose is 0.25-0.3 mol/L, 0.3-0.35 mol/L, 0.35-0.4 mol/L, 0.4-0.45 mol/L, or 0.45-0.5 mol/L.

Optionally, the volume fraction of glycerol in the adipose tissue cryopreservation protective agent is 60%-65%, 65%-70%, 70%-75%, or 75%-80%.

Optionally, the mass concentration of trehalose in the adipose tissue cryopreservation protective agent is 0.009-0.015 g/ml, 0.015-0.020 g/ml, 0.020-0.030 g/ml, 0.030-0.040 g/ml, 0.050-0.060 g/ml, 0.060-0.070 g/ml, 0.070-0.080 g/ml, 0.080-0.090 g/ml, 0.090-0.100 g/ml, 0.100-0.120 g/ml, 0.120-0.140 g/ml, 0.140-0.160 g/ml, 0.160-0.180 g/ml, or 0.180-0.200 g/ml.

The adipose tissue cryopreservation protective agent enables stable storage of adipose tissue at −196° C. for at least 6 months.

The adipose tissue cryopreservation protective agent provided by the present disclosure includes, based on the total volume of the adipose tissue cryopreservation protective agent, the following components:

glycerol with a volume fraction of 60-80%;

0.090-0.200 g/ml of trehalose; and

a PBS buffer serving as the solvent.

In one embodiment, the content of the trehalose is 0.25-0.50 mol/L.

Optionally, the volume fraction of glycerol in the adipose tissue cryopreservation protective agent is 60%-65%, 65%-70%, 70%-75%, or 75%-80%.

Optionally, the mass concentration of trehalose in the adipose tissue cryopreservation protective agent is 0.009-0.015 g/ml, 0.015-0.020 g/ml, 0.020-0.030 g/ml, 0.030-0.040 g/ml, 0.050-0.060 g/ml, 0.060-0.070 g/ml, 0.070-0.080 g/ml, 0.080-0.090 g/ml, 0.090-0.100 g/ml, 0.100-0.120 g/ml, 0.120-0.140 g/ml, 0.140-0.160 g/ml, 0.160-0.180 g/ml, or 0.180-0.200 g/ml.

The solute of PBS buffer includes: 200 mmol/L of Na₂HPO₄, 35 mmol/L of KH₂PO₄, 2.74 mol/L of NaCl and 53 mmol/L of KCI. The solvent of the PBS buffer is water. The pH of the PBS buffer is 7.2-7.6

Further, the adipose tissue cryopreservation protective agent is free of dimethyl sulfoxide, animal serum, or human albumin.

The method for preparing an adipose tissue cryopreservation protective agent according to the present disclosure includes the following operations:

1) adding trehalose into PBS buffer to prepare trehalose solution; and

2) preparing the adipose tissue cryopreservation protective agent using glycerol as the solute and the trehalose solution as the solvent.

In one embodiment, in operation 1), the concentration of trehalose in the trehalose solution is 0.25 mol/L-0.50 mol/L. Optionally, the concentration of the trehalose in the trehalose solution is 0.25-0.3 mol/L, 0.3-0.35 mol/L, 0.35-0.4 mol/L, 0.4-0.45 mol/L, or 0.45-0.5 mol/L.

In one embodiment, in operation 2), the volume fraction of glycerol in the adipose tissue cryopreservation protective agent is 60%-80%. Optionally, the volume fraction of glycerol is 60%-65%, 65%-70%, 70%-75%, or 75%-80%.

Further, the operation 1) further includes autoclaving the obtained trehalose solution.

Further, the operation 2) is carried out under aseptic conditions.

The glycerol is medically sterile glycerol.

The method for cryopreservation of adipose tissue provided by the present disclosure includes the following operations:

1) mixing the adipose tissue cryopreservation protective agent with an adipose tissue at a volume ratio of 1:1 to obtain a mixture 1;

2) placing the mixture 1 in a programmed cooling box and performing programmed cooling at −80° C.; and

3) placing the resulting mixture 1 obtained in operation 2) in liquid nitrogen for cryopreservation, to obtain a cryopreserved adipose tissue.

Further, the operation 1) further includes sealing the mixture 1.

In operation 2), the program cooling time is at least 12 hours.

The method for rewarming a cryopreserved adipose tissue provided by the present disclosure includes the following operations:

a) taking out the cryopreserved adipose tissue from liquid nitrogen, and heating the adipose tissue in water bath;

b) eluting the adipose tissue obtained in operation a) with PBS buffer, and centrifuging for a first time; and

c) collecting the upper-layer substance obtained after the centrifuging for the first time, eluting with PBS buffer, and centrifuging for a second time, and the upper-layer substance obtained after the centrifuging for the second time is the rewarmed adipose tissue.

Furthermore, in operation a), the temperature of the water bath is 37° C.

Furthermore, in operation a), the duration of the water bath is 37° C.

In one embodiment, in operation b), the duration of the eluting is 3 minutes.

In one embodiment, in operation b), the centrifugation conditions include a rotational speed of 500 rpm, and a duration of 3 minutes.

In one embodiment, in operation c), the duration of the eluting is 3 minutes.

In one embodiment, in operation c), the centrifugation conditions include a rotational speed of 500 rpm, and a duration of 3 minutes.

Neither the method for cryopreservation of adipose tissues nor the method for rewarming a cryopreserved adipose tissue according to the present disclosure involves the purpose of diagnosis and treatment. The method of the present disclosure may be used for health care purposes or for basic research purposes.

Before further describing the specific embodiments of the present disclosure, it should be understood that the scope of protection of the present disclosure is not limited to the following specific embodiments; it should also be understood that the terms used in the embodiments of the present disclosure are just for describing the specific embodiments instead of limiting the scope of the present disclosure. In the present specification and claims, the singular forms “a”, “an” and “the” include the plural forms, unless specifically stated otherwise.

When the numerical ranges are given by the embodiments, it is to be understood that the two endpoints of each numerical range and any value between the two endpoints may be selected unless otherwise stated. Unless otherwise defined, all technical and scientific terms used in the present disclosure have the same meaning as commonly understood by one skill in the art. In addition to the specific method, equipment and material used in the embodiments, any method, equipment and material in the existing technology similar or equivalent to the method, equipment and material mentioned in the embodiments of the present disclosure may be used to realize the invention according to the grasp of the existing technology by those skilled in the art and the record of the invention.

Unless otherwise stated, the experimental methods, detection methods, and preparation methods disclosed in the present disclosure all employ conventional techniques of molecular biology, biochemistry, chromatin structure and analysis, analytical chemistry, cell culture, and recombinant DNA technology in the technical field and related fields.

### Embodiment 1

The adipose tissue cryopreservation protective agent is prepared according to the following formula:

glycerol with a volume fraction of 60%;

0.090 g/ml of trehalose; and

a PBS buffer serving as the solvent.

### Embodiment 2

1. After the adipose tissue was separated under aseptic conditions, the adipose tissue was mixed uniformly with the adipose tissue cryopreservation protective agent described in Embodiment 1 at a volume ratio of 1:1, and the cryopreservation tube containing the uniformly mixed mixture was turned upside down for mixing well.

2. The cryopreservation tube was placed in a programmed cooling box, which was then placed in a refrigerator at −80° C. After cooling for 12 hours, the cryopreservation tube was transferred to liquid nitrogen at −196° C.

3. When it is necessary to use the cryopreserved adipose tissue, taking out the cryopreservation tube from the liquid nitrogen and placing the cryopreservation tube in a 37° C. water bath for 2 minutes for rewarming.

4. The cryopreservation tube was transferred to aseptic conditions, the mixture in the cryopreservation tube was placed in an aseptic centrifuge tube, the mixture was first eluted with PBS buffer for 3 minutes and then centrifuged at 500 rpm for 3 minutes. The lower-layer liquid obtained after the centrifugation was discarded. The upper-layer substance was eluted with PBS buffer for 3 minutes and then centrifuged at 500 rpm for 3 minutes. The lower-layer liquid obtained after the centrifugation was discarded.

### Embodiment 3

The adipose tissues were subjected to the procedures of programmed cooling and preservation at −80° C. or −196° C. for 6 months after being added with the following, respectively: no cryopreservation protective agent, FBS+DMSO protective agent (10%DMSO+90%FBS), trehalose protective agent (0.25 mol/L trehalose solution, with the solvent being phosphate buffer solution having a formulation of: 200 mmol/L Na₂HPO₄, 35 mmol/L KH₂PO₄, 2.74 mol/L NaCl, 53 mmol/L KCI, pH 7.2-7.6), and the adipose tissue cryopreservation protective agent as described in Embodiment 1. The adipose tissues were taken out, and the adipose transplantation was performed on the back of nude mice. As shown in FIG. 2, the adipose tissues were transplanted on both sides of the back in the middle part of the mouse body, and 0.2 ml of adipose tissues was injected on each side. After observing for one month, the remaining tissue was taken out and weighed. The retention rate was calculated. (Retention rate=mass of remaining tissue±mass of transplanted tissue*100%).

The retention rate results are shown in FIG. 4. It can be seen from the figures that under two temperature conditions, the retention rate of the present disclosure is better than that of the traditional cryopreservation protective agent and the group without a protective agent. The results indicate that after the use of the present disclosure, the postoperative retention rate, which is the most important indicator in the clinical practice of adipose transplantation, is significantly improved, implying that the present disclosure has a high clinical application value.

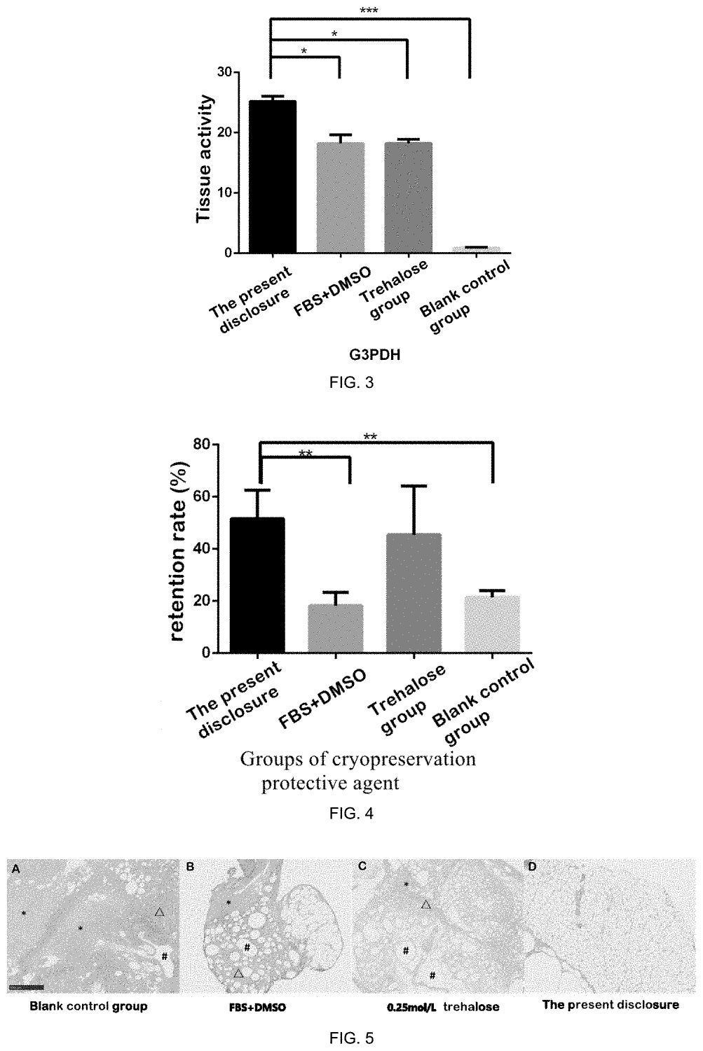

G3PDH assay was performed on the adipose tissues using Glycerol-3-Phosphate Dehydrogenase (G3PDH) Assay Kit (Sigma-Aldrich, MAK208). The experiment was carried out according to the instructions of the kit. As shown in FIG. 3, G3PDH is an assay for quantitative measurement of tissue block activity. For the same mass of sample, the larger the G3PDH value, the higher the biological activity within the tissue block. Based on experiments, it was verified that the adipose tissue preserved by the adipose tissue cryopreservation protective agent of the present disclosure had better activity than those preserved by the traditional cryopreservation protective agent and the protective agent containing only trehalose, and there was a statistical difference.

### Embodiment 4

The adipose tissues cryopreserved at −196° C. for 6 months after being added without any cryopreservation protective agent, added with FBS+DMSO protective agent, added with trehalose protective agent, or added with the cryopreservation protective agent as described in Embodiment 1 were transplanted to the back of nude mice. After one month of observation, the transplanted adipose tissues were taken out, sliced, and stained with hematoxylin-eosin (HE) for observation.

As shown in FIG. 5, the adipose tissues were transplanted to the back of nude mice after being cryopreserved at −196° C. for 6 months. After one month of observation, the transplanted adipose tissues were taken out, sliced, and stained with HE for observation.

It can be seen that there were more fibrosis tissues in the blank control, traditional group (FBS+DMSO) and trehalose groups, indicating more necrotic tissues after transplantation. There were more inflammatory exudates in these three groups, indicating that both low temperature and these cryopreservation protective agents produced great inflammatory stimulation on the adipose tissues. There were more fat vacuoles in these three groups, indicating that more lipid droplets ruptured after cryopreservation, producing non-functional vacuoles. In contrast, after the use of the protective agent of the present disclosure, no obvious fibrosis or inflammatory exudates were seen after adipose tissue transplantation, the adipose tissues had fewer vacuoles and intact tissue structure, indicating a better protective effect.

Based on comparison, it can be seen that, under the same conditions, the cryopreservation protective agent described in Embodiment 1 produced significantly less vacuoles and fibrosis areas than the traditional protective agent and the blank control group, demonstrating that the cryopreservation protective agent of the present disclosure is superior to the traditional protective agent in terms of both effectiveness and quality for long-term cryopreservation of adipose tissue for transplantation.

### Embodiment 5

The adipose tissue cryopreservation protective agent is prepared according to the following formula:

glycerol with a volume fraction of 80%;

0.200 g/ml of trehalose; and

a PBS buffer serving as the solvent.

### Embodiment 6

The adipose tissue cryopreservation protective agent is prepared according to the following formula:

glycerol with a volume fraction of 70%;

0.100 g/ml of trehalose; and

a PBS buffer serving as the solvent.

### Embodiment 7

The adipose tissue cryopreservation protective agent is prepared according to the following formula:

glycerol with a volume fraction of 65%;

0.150 g/ml of trehalose; and

a PBS buffer serving as the solvent.

### Embodiment 8

The adipose tissue cryopreservation protective agent is prepared according to the following formula:

glycerol with a volume fraction of 68%;

0.020 g/ml of trehalose; and

a PBS buffer serving as the solvent.

### Embodiment 9

The adipose tissue cryopreservation protective agent is prepared according to the following formula:

glycerol with a volume fraction of 72%;

0.030 g/ml of trehalose; and

a PBS buffer serving as the solvent.

### Embodiment 10

The adipose tissue cryopreservation protective agent is prepared according to the following formula:

glycerol with a volume fraction of 78%;

0.040 g/ml of trehalose; and

a PBS buffer serving as the solvent.

### Embodiment 11

The adipose tissue cryopreservation protective agent is prepared according to the following formula:

glycerol with a volume fraction of 80%;

0.160 g/ml of trehalose; and

a PBS buffer serving as the solvent.

The present disclosure was also validated in an animal model using the adipose tissue cryopreservation protective agents of the present disclosure, and results showed that after using the present disclosure for adipose tissue cryopreservation, the retention rate of the transplanted adipose was significantly improved, and the tissue morphology was more complete than that treated with the traditional protective agent, with significantly less inflammatory infiltration, fat vacuoles and fibrotic tissue. It was demonstrated that the adipose tissue treated with the present disclosure was also superior to those treated with traditional group and the blank control group in terms of transplantation quality.

The above embodiments are intended to illustrate the disclosed embodiments of the present disclosure and should not be understood as restrictions on the present disclosure. In addition, various modifications of the present disclosure, as well as variations of the methods and compositions of the disclosure, will be apparent to those skilled in the art without departing from the scope of the present disclosure. While the disclosure has been described in detail in connection with various specific preferred embodiments thereof, however, it should be understood that the present disclosure should not be limited to these specific embodiments. In fact, various modifications to the present disclosure as apparent to those skilled in the art are intended to be included within the scope of the present disclosure.

1. Use of glycerol in preparing an adipose tissue cryopreservation protective agent.

2. The use according to claim 1, wherein glycerol is the active ingredient of the adipose tissue cryopreservation protective agent.

3. The use according to claim 1, wherein the adipose tissue cryopreservation protective agent is free of dimethyl sulfoxide, animal serum, or human albumin.

4. The use according to claim 1, wherein the adipose tissue cryopreservation protective agent further comprises trehalose and PBS buffer.

5. The use according to claim 4, wherein the adipose tissue cryopreservation protective agent comprises, based on the total volume of the adipose tissue cryopreservation protective agent, the following components:
glycerol with a volume fraction of 60-80%;
0.090-0.200 g/ml of trehalose; and
a PBS buffer serving as the solvent.

6. The use according to claim 1, wherein the adipose tissue cryopreservation protective agent enables stable storage of adipose tissue at −196° C. for at least 6 months.

7. An adipose tissue cryopreservation protective agent, comprising, based on the total volume of the adipose tissue cryopreservation protective agent, the following components:
glycerol with a volume fraction of 60-80%;
0.090-0.200 g/ml of trehalose; and
a PBS buffer serving as the solvent.

8. The adipose tissue cryopreservation protective agent according to claim 7, wherein the adipose tissue cryopreservation protective agent is free of dimethyl sulfoxide, animal serum, or human albumin.

9. The adipose tissue cryopreservation protective agent according to claim 7, wherein the adipose tissue cryopreservation protective agent enables stable storage of adipose tissue at −196° C. for at least 6 months.

10. A method for preparing an adipose tissue cryopreservation protective agent, comprising the following operations:
1) adding trehalose into PBS buffer to prepare trehalose solution; and
2) preparing the adipose tissue cryopreservation protective agent using glycerol as the solute and the trehalose solution as the solvent.

11. The method according to claim 10, further comprising one or more of the following:
a. in operation 1), a concentration of trehalose in the trehalose solution ranges from 0.25 mol/L to 0.50 mol/L;
b. in operation 2), a volume fraction of glycerol in the adipose tissue cryopreservation protective agent is 60%-80%.

12. A method for cryopreservation of adipose tissue, comprising the following operations:
1) mixing the adipose tissue cryopreservation protective agent according to claim 7 with an adipose tissue at a volume ratio of 1:1 to obtain a mixture 1;
2) placing the mixture 1 in a programmed cooling box and performing programmed cooling at −80° C.; and
3) placing the resulting mixture 1 obtained in operation 2) in liquid nitrogen for cryopreservation, to obtain a cryopreserved adipose tissue.

13. A method for rewarming a cryopreserved adipose tissue, comprising the following operations:
a) taking out the cryopreserved adipose tissue obtained in operation 3) of claim 12 from liquid nitrogen, and heating the adipose tissue in water bath;
b) eluting the adipose tissue obtained in operation a) with PBS buffer, and centrifuging for a first time; and
c) collecting the upper-layer substance obtained after the centrifuging for the first time, eluting with PBS buffer, and centrifuging for a second time, and the upper-layer substance obtained after the centrifuging for the second time is a rewarmed adipose tissue.

14. The use according to claim 1, further comprising the following operations:
1) adding trehalose into PBS buffer to prepare trehalose solution; and
2) preparing the adipose tissue cryopreservation protective agent using glycerol as the solute and the trehalose solution as the solvent.
